(19) United States
(12) Patent Application Publication
(10) Pub. No.: US 2026/0070958 A1
(43) Pub. Date: Mar. 12, 2026

(54) METHODS AND MATERIALS FOR TREATING CANCER

(71) Applicant: Mayo Foundation for Medical Education and Research, Rochester, MN (US)

(72) Inventors: Saad J. Kenderian, Rochester, MN (US); Stephen J. Russell, Rochester, MN (US)

(21) Appl. No.: 19/062,838

(22) Filed: Feb. 25, 2025

Related U.S. Application Data
(62) Division of application No. 17/287,164, now Pat. No. 12,264,189.
(60) Provisional application No. 62/753,732, filed on Oct. 31, 2018.

(51) Int. Cl. C07K 14/705; A61K 38/00; A61K 40/11; A61K 40/31; A61K 40/42; A61P 35/00; C07K 16/28; C12N 7/00

(52) U.S. Cl. CPC C07K 14/70503; A61K 40/11; A61K 40/31; A61K 40/4211; A61P 35/00; C07K 16/2809; C12N 7/00; A61K 38/00; A61K 2239/48

(57) ABSTRACT

This document provides methods and materials for generating T cells expressing a selected antigen receptor (e.g., a chimeric antigen receptor) in vivo. For example, viral particles containing nucleic acid encoding a selected antigen receptor, cells capable of producing such viral particles, and methods for administering such viral particles and/or cells to a mammal to generate T cells expressing that selected antigen receptor within the mammal are provided.

### CROSS-REFERENCE TO RELATED APPLICATIONS

This application is a continuation of U.S. application Ser. No. 17/287,164, filed on Apr. 21, 2021, which is a National Stage application under 35 U.S.C. § 371 of International Application No. PCT/US2019/059260, having an International Filing Date of Oct. 31, 2019, which claims priority to U.S. Application Ser. No. 62/753,732, filed on Oct. 31, 2018. The disclosure of the prior applications are considered part of the disclosure of this application, and are incorporated in their entirety into this application.

### SEQUENCE LISTING

This application contains a Sequence Listing that has been submitted electronically as an XML file named 07039-1816002_SL_ST26.xml. The XML file, created on Feb. 25, 2025, is 11,444 bytes in size. The material in the XML file is hereby incorporated by reference in its entirety.

### BACKGROUND

### 1. Technical Field

This document relates to methods and materials for generating T cells expressing an antigen receptor (e.g., a chimeric antigen receptor) in vivo. For example, viral particles including nucleic acid encoding an antigen receptor can be administered to a mammal to generate T cells expressing that antigen receptor within the mammal. In some cases, in vivo generation of T cells expressing a selected antigen receptor can be used to target cells (e.g., cancer cells) presenting an antigen (e.g., a tumor antigen) recognized by that selected antigen receptor.

### 2. Background Information

Chimeric antigen receptor T (CART) cell therapy is a revolutionary therapy in the treatment of cancer. In B cell malignancies, CD19 directed CART cell therapy resulted in unprecedented responses that led to FDA approval of two products in 2017. However, CART cell therapies are limited by complexity, high costs, the requirement for elaborate protocols that can only be applied in specialized good manufacturing practices (GMP) grade facilities, the highly individualized process, and the lead-time required for CART cell manufacturing. For example, in CART cell therapy, patients undergo apheresis, and T cells are isolated, stimulated, transduced, and expanded ex vivo for a period of 7-10 days to generate CART cells. Then patients receive lympho-depleting chemotherapy followed by infusion of the generated CART cells. Further, the activity of CART cell therapy in solid tumors is limited.

### SUMMARY

This document provides methods and materials for generating T cells expressing a selected antigen receptor (e.g., a chimeric antigen receptor) in vivo.

In some cases, one or more viral particles containing nucleic acid encoding a selected antigen receptor can be administered to a mammal to generate T cells expressing that selected antigen receptor within the mammal. For example, one or more viral particles containing nucleic acid encoding a chimeric antigen receptor (CAR) administered to a mammal can transduce one or more T cells within the mammal (e.g., to introduce the nucleic acid encoding the selected antigen receptor into the T cells such that the T cells express the CAR) to generate one or more CART cells within the mammal.

In some cases, one or more cells capable of producing one or more viral particles containing nucleic acid encoding a selected antigen receptor (also referred to as virus-producing cells) can be administered to a mammal to generate T cells expressing that selected antigen receptor within the mammal. For example, one or more cells capable of producing viral particles containing nucleic acid encoding a CAR administered to a mammal can result in the production of infectious viral particles, which in turn can transduce one or more T cells within the mammal (e.g., to introduce the nucleic acid encoding the selected antigen receptor into the T cells such that the T cells express the CAR) to generate one or more CART cells within the mammal.

In some cases, in vivo generation of one or more T cells expressing a selected antigen receptor can be used to target cells (e.g., cancer cells) presenting an antigen (e.g., a tumor antigen) recognized by the selected antigen receptor. As described herein, one or more viral particles containing nucleic acid encoding a CAR (or one or more cells capable of producing such viral particles) can be administered to a mammal having cancer (e.g., a cancer presenting a tumor antigen) to generate one or more CART cells that can target cancer cells within the mammal. In some cases, the methods and materials described herein can be used for in vivo generation of tumor specific cytotoxic T cells (CTLs).

The ability to generate tumor-specific CARTs in vivo provides a unique opportunity to bypass ex vivo CART cell production, the most time consuming, labor intensive, and costly portion of CART cell manufacturing. The ability to generate tumor-specific CARTs in vivo also provides a unique opportunity to generate CART cells capable of targeting (e.g., locating and destroying) cancer cells, including cancer cells in solid tumors (that are typically undetectable by the immune system) and cancer cells at secondary (e.g., metastatic) locations. In addition, CART cells generated in vivo as described herein (e.g., by administering one or more viral particles containing nucleic acid encoding a selected antigen receptor or by administering one or more cells capable of producing such viral particles) can be more effective than ex vivo expanded and transduced T cells because a T cell generated in vivo as described herein can be naturally activated and differentiated in the tumor (or in the region of the tumor) and/or lymph nodes. Further, the methods and materials described herein can be more conducible to “off the shelf” reagents, thereby allowing individualized therapies (e.g., in the form of tumor-specific immune responses) to be quickly and readily applied to wide patient populations while limiting costs.

In general, one aspect of this document features a method for generating T cells expressing a selected antigen receptor in vivo within a mammal. The method comprises, or consists essentially of, administering a viral particle to the mammal. The viral particle comprises, or consists essentially of, (a) nucleic acid encoding the selected antigen receptor and (b) an exogenous molecule that targets the viral particle to infect T cells within the mammal, wherein the viral particle infects T cells comprising an endogenous T cell receptor within the mammal to form infected T cells, and wherein the infected T cells express the selected antigen receptor and the endogenous T cell receptor. The mammal can be a human. The viral particle can be a lentiviral particle. The antigen receptor can be a chimeric antigen receptor. The viral particle can target one or more cancer cells. The one or more cancer cells can express a tumor-specific antigen. The selected antigen receptor can target the tumor-specific antigen. The tumor-specific antigen can be CD19. The tumor-specific antigen can be CD19, and the antigen receptor targeting the tumor-specific antigen can be a CAR targeting CD19. The nucleic acid encoding the CAR targeting CD19 can comprise a nucleic acid sequence set forth in SEQ ID NO:1. The exogenous molecule can comprise an antigen-binding domain that can recognize CD3, and the viral particle can further comprise nucleic acid encoding the antigen-binding domain that can recognize CD3. The antigen-binding domain can be a single-chain variable fragment. The nucleic acid encoding the single-chain variable fragment that can recognize CD3 can comprise a nucleic acid sequence set forth in SEQ ID NO:2. The administration can be intravenous injection or intratumoral injection.

In another aspect, this document features a method for generating T cells expressing a selected antigen receptor in vivo within a mammal. The method comprises, or consists essentially of, administering, to the mammal, a cell capable of producing a viral particle, wherein the viral particle comprises, or consists essentially of, (a) nucleic acid encoding the selected antigen receptor, and (b) an exogenous molecule that targets the viral particle to infect T cells within the mammal, wherein the viral particle is produced within the mammal and infects T cells comprising an endogenous T cell receptor within the mammal to form infected T cells, and wherein the infected T cells express the selected antigen receptor and the endogenous T cell receptor. The mammal can be a human. The viral particle can be a lentiviral particle. The antigen receptor can be a chimeric antigen receptor. The viral particle can target one or more cancer cells. The one or more cancer cells can express a tumor-specific antigen. The selected antigen receptor can target the tumor-specific antigen. The tumor-specific antigen can be CD19. The tumor-specific antigen can be CD19, and the antigen receptor targeting the tumor-specific antigen can be a CAR targeting CD19. The nucleic acid encoding the CAR targeting CD19 can comprise a nucleic acid sequence set forth in SEQ ID NO:1. The exogenous molecule can comprise an antigen-binding domain that can recognize CD3, and the viral particle can further comprise nucleic acid encoding the antigen-binding domain that can recognize CD3. The antigen-binding domain can be a single-chain variable fragment. The nucleic acid encoding the single-chain variable fragment that can recognize CD3 can comprise a nucleic acid sequence set forth in SEQ ID NO:2. The administration can be intravenous injection or intratumoral injection.

In another aspect, this document features a method for treating a mammal having cancer comprising cancer cells presenting a selected antigen. The method comprises, or consists essentially of, administering a viral particle to the mammal, wherein the viral particle comprises, or consists essentially of, (a) nucleic acid encoding an antigen receptor targeting the selected antigen, and (b) an exogenous molecule that targets the viral particle to infect T cells within the mammal, wherein the administration is effective to generate T cells expressing the antigen receptor in vivo. The mammal can be a human. The viral particle can be a lentiviral particle. The cancer can be a B cell cancer. The antigen receptor can be a chimeric antigen receptor. The cancer can comprise cancer cells expressing a tumor-specific antigen. The antigen receptor can target the tumor-specific antigen. The tumor-specific antigen can be CD19. The tumor-specific antigen can be CD19, and the antigen receptor targeting the tumor-specific antigen can be a CAR targeting CD19. The nucleic acid encoding the CAR targeting CD19 can comprise a nucleic acid sequence set forth in SEQ ID NO: 1. The exogenous molecule can comprise an antigen-binding domain that can recognize CD3, and the viral particle further can comprise nucleic acid encoding the antigen-binding domain that can recognize CD3. The antigen-binding domain can be a single-chain variable fragment. The nucleic acid encoding the single-chain variable fragment that can recognize CD3 can comprise a nucleic acid sequence set forth in SEQ ID NO:2. The administration can be intratumoral injection.

In another aspect, this document features a method for treating a mammal having cancer comprising cancer cells presenting a selected antigen. The method comprises, or consists essentially of, administering a cell producing a viral particle to the mammal, wherein the viral particle comprises, or consists essentially of, (a) nucleic acid encoding an antigen receptor targeting the selected antigen, and (b) an exogenous molecule that targets the viral particle to infect T cells within the mammal, wherein the administration is effective to generate T cells expressing the antigen receptor in vivo. The mammal can be a human. The viral particle can be a lentiviral particle. The cancer can be a B cell cancer. The antigen receptor can be a chimeric antigen receptor. The cancer can comprise cancer cells expressing a tumor-specific antigen. The antigen receptor can target the tumor-specific antigen. The tumor-specific antigen can be CD19. The tumor-specific antigen can be CD19, and the antigen receptor targeting the tumor-specific antigen can be a CAR targeting CD19. The nucleic acid encoding the CAR targeting CD19 can comprise a nucleic acid sequence set forth in SEQ ID NO:1. The exogenous molecule can comprise an antigen-binding domain that can recognize CD3, and the viral particle further can comprise nucleic acid encoding the antigen-binding domain that can recognize CD3. The antigen-binding domain can be a single-chain variable fragment. The nucleic acid encoding the single-chain variable fragment that can recognize CD3 can comprise a nucleic acid sequence set forth in SEQ ID NO:2. The administration can be intratumoral injection.

In another aspect, this document features a method for generating T cells expressing a selected antigen receptor in vivo within a mammal. The method comprises (or consists essentially of or consists of) administering, to the mammal, a cell capable of producing a viral particle, wherein the viral particle comprises nucleic acid encoding the selected antigen receptor, wherein the viral particle is produced within the mammal and infects T cells comprising an endogenous T cell receptor within the mammal to form infected T cells, and wherein the infected T cells express the selected antigen receptor and the endogenous T cell receptor. The mammal can be a human. The viral particle can be a lentiviral particle. The antigen receptor can be a chimeric antigen receptor. The viral particle can target one or more cancer cells. The one or more cancer cells can express a tumor-specific antigen. The selected antigen receptor can target the tumor-specific antigen. The tumor-specific antigen can be CD19. The antigen receptor targeting the tumor-specific antigen can be a CAR targeting CD19. The nucleic acid encoding the CAR targeting CD19 can comprise a nucleic acid sequence set forth in SEQ ID NO:1. The exogenous molecule can comprise an antigen-binding domain that can recognize CD3 and wherein the viral particle further comprises nucleic acid encoding the antigen-binding domain that can recognize CD3. The antigen-binding domain can be a single-chain variable fragment. The nucleic acid encoding the single-chain variable fragment that can recognize CD3 can comprise a nucleic acid sequence set forth in SEQ ID NO:2. The administration can be an intravenous injection or an intratumoral injection. The nucleic acid encoding the selected antigen receptor can comprise nucleic acid encoding a scFv set forth in Table 1.

In another aspect, this document features a method for treating a mammal having cancer comprising cancer cells expressing a selected antigen. The method comprises (or consists essentially of or consists of) administering a cell capable of producing a viral particle to the mammal, wherein the viral particle comprises nucleic acid encoding an antigen receptor targeting the selected antigen, wherein the administration is effective to generate T cells expressing the antigen receptor in vivo. The mammal can be a human. The viral particle can be a lentiviral particle. The cancer can be a B cell cancer. The antigen receptor can be a chimeric antigen receptor. The nucleic acid encoding the antigen receptor can comprise nucleic acid encoding a scFv set forth in Table 1. The administration can be an intravenous injection or an intratumoral injection.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIGS. 1A-1B contain schematic diagrams of exemplary methods for in vivo generation of CART cells. In FIG. 1A, viral particles containing nucleic acid encoding CARs are delivered intravenously or by intratumoral injection to a tumor site. The viral particles can include one or more targeting molecules (e.g., a single chain antibody that binds to CD3) that help direct the viral particles to infect T cells in vivo. In FIG. 1B, cells capable of producing viral particles containing nucleic acid encoding CARs are delivered by intratumoral injection to a tumor site. The produced viral particles can include one or more targeting molecules (e.g., a single chain antibody that binds to CD3) that help direct the produced viral particles to infect T cells in vivo.

FIG. 2 shows an exemplary nucleic acid sequence (SEQ ID NO:1) encoding a CAR targeting a cluster of differentiation 19 (CD19) antigen.

FIG. 3 shows an exemplary nucleic acid sequence (SEQ ID NO:2) encoding a modified viral envelope polypeptide capable of target a cluster of differentiation 3 (CD3) antigen.

FIGS. 4A-4B. Virus producing cells are capable of transducing T cells and generating CART cells. FIG. 4A: Titration Assay of virus producing cells to T cells. Approximately 5:1 virus producing cell: T cell is used to achieve sufficient transduction of T cells and generation of CART cells. FIG. 4B: Cytotoxicity Assay. CART cells generated through 293 T cell direct transduction of T cells exhibited potent anti-tumor activity against the CD19+ cell line NALM6 but not the CD19 negative cell line MOLM13.

FIGS. 5A-5F. (A) Schema of the experiment. NSG mice were engrafted with the human CD19⁺, luc⁺ NALM6 cell line subcutaneously. Engraftment was confirmed by bioluminescent imaging, and mice were then randomized to receive either ex-vivo expanded human CART19 or virus producing 293T cells transfected with human CAR19 plasmid followed by normal T cells intravenously. (B) Peripheral blood sampling 10 days later shows CART19 generation. Flow blots of human CD45⁺ cells: CD3 on the X axis, and CAR on the Y axis. (C) Percentage of CAR⁺ T cells detected in peripheral blood was higher when 293T cells were injected IV with T cells compared to ex-vivo transduced CART19 cells. (D) Antitumor activity after in vivo generation of CART19 cells was similar to that observed using ex vivo generated CART19. Untreated mice died of the disease within 20 days. (E) Mice treated with either (1) ex vivo generated CART19 or (2) 293T cells plus T cells (in vivo generated CART19) exhibited similar antitumor control on day 14 and day 19. (F) Mice treated with either (1) ex vivo generated CART19 or (2) 293T cells plus T cells (in vivo generated CART19) exhibited similar overall survival that is improved compared to untreated mice.

### DETAILED DESCRIPTION

This document provides methods and materials for generating T cells expressing a selected antigen receptor (e.g., a CAR) in vivo. In some cases, one or more viral particles containing (e.g., engineered to contain) nucleic acid encoding a selected antigen receptor (e.g., a selected CAR) can be administered to a mammal to generate T cells in vivo that express that selected antigen receptor. For example, one or more viral particles containing nucleic acid encoding a CAR can be administered to a mammal where the viral particle(s) can transduce one or more T cells such that the T cell(s) express the nucleic acid encoding the selected CAR, thereby generating CART cells expressing the selected antigen receptor within the mammal. In some cases, one or more cells capable of producing one or more viral particles containing (e.g., engineered to contain) nucleic acid encoding a selected antigen receptor (e.g., a selected CAR) can be administered to a mammal to generate T cells in vivo that express that selected antigen receptor. For example, one or more cells capable of producing viral particles containing nucleic acid encoding a CAR can be administered to a mammal such that the cell(s) produce infectious viral particles containing nucleic acid encoding the CAR, which in turn can transduce one or more T cells such that the T cell(s) express the nucleic acid encoding the selected CAR, thereby generating CART cells expressing the selected antigen receptor within the mammal.

In some embodiments, this document provides viral particles containing nucleic acid encoding a selected antigen receptor. A viral particle described herein (e.g., a viral particle containing nucleic acid encoding a selected antigen receptor) can include viral components (e.g., genetic material (e.g., a viral genome), a capsid, and/or an envelope) from any appropriate virus. A virus can be an infectious virus. A virus can be a chimeric virus. A virus can be a recombinant virus. In some cases, a viral particle can include viral components from the same virus. In some cases, a viral particle can be a recombinant viral particle. For example, a recombinant viral particle can include viral components from different viruses (e.g., two or more different viruses). Examples of viruses from which viral components can be obtained include, without limitation, retroviruses, (e.g., lentiviruses), measles viruses, canine distemper viruses, Cedar viruses, and Nipah viruses. For example, a viral particle can include a lentiviral backbone and a measles virus envelope.

A viral particle described herein (e.g., a viral particle containing nucleic acid encoding a selected antigen receptor) can contain (e.g., can be engineered to contain) nucleic acid encoding any appropriate antigen receptor. In some cases, an antigen receptor can be a heterologous antigen receptor. In some cases, an antigen receptor can be a CAR. In some cases, an antigen receptor can be a tumor antigen receptor (e.g., an antigen receptor that targets a tumor antigen). In cases where a viral particle contains nucleic acid encoding a tumor antigen receptor, the tumor antigen receptor can target any appropriate tumor antigen. In some cases, a tumor antigen can be a cell surface tumor antigen. In some cases, a tumor antigen can be a tumor-associated antigen (TAA; e.g., an antigen, such as an abnormal protein, present on tumor cells). In some cases, a tumor antigen can be a tumor-specific antigen (TSA; e.g., an antigen present only on tumor cells). Examples of tumor antigens that can be targeted by a selected antigen receptor include, without limitation, CD19, mucin 1 (MUC-1), human epidermal growth factor receptor 2 (HER-2), estrogen receptor (ER), epidermal growth factor receptor (EGFR), folate receptor alpha, mesothelin, alphafetoprotein (AFP), carcinoembryonic antigen (CEA), CA-125, epithelial tumor antigen (ETA), melanoma-associated antigen (MAGE), membrane cofactor protein 11 (MCP11, also known as CD46), CD123, CD33, CLEC12A, Mesothelin, CD22, CD20, BCMA, CS-1, CD138, CD44v6, NKGD2A, and CD38. In some cases, an antigen receptor can be a disease-associated antigen receptor (e.g., a disease-associated antigen that is associated with a disease other than cancer). In cases where a viral particle contains nucleic acid encoding a disease-associated antigen receptor, the disease-associated antigen receptor can target any appropriate disease-associated antigen. In some cases, a viral particle described herein can contain nucleic acid encoding a CAR targeting CD19 (e.g., an anti-CD19 CAR). Examples of scFv sequences and other binding sequences that can be used to design a CAR to a particular tumor antigen include, without limitation, those set forth in Table 1.

TABLE 1
Representative scFv antibodies and sequences for targeting
CARs to tumor antigens.

| Antigen | scFv antibody name | Reference(s) |
|---|---|---|
| CD19 | FMC63 | Milone et al., Mol. Ther., 17(8):1453-64 (2009) (PMID: 26330164) |
| | MOR208 | Kellner et al., Leukemia, 27(7):1595-8 (2013) (FWD: 23277329) |
| | Humanized scFv | International Patent Application Publication No. WO2015157252 |
| | 4G7 | EP Patent No. EP2997141 |
| | Low affinity scFv | Chinese Patent No. CN107406517 |
| MUC-1 | 5E5 | Posey et al., Immunity, 45(5):947-948 (2016) (PMID: 27851918) |
| HER-2 | 4D5 | Ohnishi et al., Br. J. Cancer, 71(5):969-73 (1995) (PMID: 7734322) |
| | | Forsberg et at, Cancer Res., 79(5):899-904 (2019) (PMID: 30622115) |
| | | Nellan et al., J. Immunother. Cancer, 6(1):30 (2018) (PMID: 29712574) |
| | | Priceman et al., Clin Cancer Res., 24(1):95-105 (2018) |
| | | (PMID: 29061641) |
| | FRP5 | Bielamowics et al., Neuro. Oncol., 20(4):506-518 (2018) |
| | | (PMID: 29016929) |
| EGFR | M27 | Jiang et al., Cancer Immunol. Res., 6(11):1314-1326 (2018) |
| | | (PMID: 30201736) |
| | Cetuximab | Caruso et al., Cancer Res., 75(17):3505-18 (2015) (PMID: 26330164) |
| Folate receptor alpha | C4 based | Ao et al., J. Immunother., 42(8):284-296 (2019) (PMID: 31261167) |
| | MOv19 | Song et al., J. Hematol. Oncol., 9(1):56 (2016) (PMID: 27439908) |
| Mesothelin | SS1 | Haas et al., Mol. Ther., S1525-0016(19)30328-4 (2019) |
| | | (PMID: 31420241) |
| | M clone | Adusumilli et al., Sci. Trarisl. Med., 6(261):261ra151 (2014) |
| | | (PMID: 25378643) |
| AFP | ET1402L1 | Liu et al., Clin. Cancer Res., 23(2):478-488 (2017) (PMID: 27535982) |
| CEA | Anti-CEA scFv | Chi et al., Cancer Med., 8(10):4753-4765 (2019) (PMID: 31237116) |
| | CEACAM5 | Thistlethwaite et at, Cancer Immunol. Immunother., 66(11):1425-1436 |
| | | (2017) (PMID: 28660319) |
| | hMN14 | Katz et al., Clin. Cancer Res., 21(14):3149-59 (2015) |
| | | (PMID: 25850950) |
| CD123 | 22172, 22176 | Gill et al., Blood, 123(15):2343-54 (2014) (PMID: 24596416) |
| | Humanized scFv | US Patent Application Publication No. 2016/0068601 |
| | Humanized scFv | EP Patent No. EP2968415 |
| | Tagrazofusp based | Pemmaraju et al. N. Engl. J. Med., 380(17):1628-1637 (2019) |
| | | (PMID: 31018069) |
| CD33 | MY96 | Kenderian et at, Leukemia, 29(8):1637-47 (2015) (PMID: 25721896) |
| | Humanized scFv | International Patent Application Publication No. WO2016014576 |
| CLEC12A | Humanized scFv | US Patent Application Publication No. US20160051651 |
| | CLL1 scFv | International Patent Application Publication No. WO2016120219 |
| CD22 | M0971 | Fry et at, Nat. Med., 24(1):20-28 (2018) (PMID: 29155426) |
| | Humanized scFv | International Patent Application Publication No. WO/2016/164731 |
| | clones | |
| | Inotuzumab based | Kantarjian et at, N. Engl. J. Med., 375(8):740-53 (2016) |
| | | (PMID: 27292104) |
| | Moxetumomab based | Kreitman et al., Leukemia, 32(8):1768-1777 (2018) (PMID: 30030507) |
| CD20 | Rituximab | Zhang et al., Signal Transduct. Target Ther., 1:16002 (2016) |
| | | (PMID: 29263894) |
| | Leu 16 | Lee et al., J. Immunother., 41(1):19-31 (2018) (PMID: 29176334) |
| | CD20 scFvs | International Patent Application Publication No. WO/2016/164731 |
| BCMA | BCMA-02 | Raje et al., N. Engl. J. Med., 380(18):1726-1737 (2019) |
| | | (PMID: 31042825) |
| | LCAR38 | Zhao et al., J. Hematol. Oncol., 11(1):141 (2018) (PMID: 30572922) |
| | BCMA scFv | Smith et al., Cancer Immunol. Res., 7(7):1047-1053 (2019) |
| | | (PMID: 31113804) |
| | Biepitopic | Xu et al., Proc. Natl. Acad. Sci. USA, 116(19):9543-9551 (2019) |
| | | (PMID: 30988175) |
| | NVS BCMA | Cohen et at, J. Clin. Invest., 129(6):2210-2221 (2019) |
| | | (PMID: 30896447) |
| CS-1 | CS1R | Wang et al., Clin. Cancer Res., 24(1):106-119 (2018) |
| | | (PMID: 29061640) |
| | CS1 ScFv | Chu et al., Leukemia, 28(4):917-27 (2014) (PMID: 24067492) |
| | Elotuzumab based | Dimopoulos et al., N. Engl. J. Med., 379(19):1811-1822 (2018) |
| | | (PMID: 30403938) |
| CD138 | ScFv | Sun et al., Oncotarget., 10(24):2369-2383 (2019) (PMID: 31040928) |
| CD44v6 | Humanized scFv | Leuci et al., Oncoimmunology, 7(5):e1423167 (2018) |
| | | (PMID: 29721373) |
| | cMAb U36 | Sandstrom et al., Int. J. Oncol., 40(5):1525-32 (2012) |
| | | (PMID: 22307465) |
| NKG2D | scFv | Yang et al., J. Immunother. Cancer, 7(1):171 (2019) (PMID: 31288857) |
| | NKG2Dg scFv | Parihar et al., Cancer Immunol. Res., 7(3):363-375 (2019) |
| | | (PMID: 30651290) |
| CD38 | Nanobody CD38 | An et al., Mol. Pharm., 15(10):4577-4588 (2018) (PMID: 30185037) |
| | Humanized scFv | Yoshida et al., Clin. Transl. Immunology, 5(12):e116 (2016) |
| | | (PMID: 28090317) |
| GPRC5D | Humanized scFv | Smith et al., Sci. Transl. Med., 11(485)eaau7746 (2019) |
| | | (PMID: 30918115) |
| CD79b | scFv (L-H) and (H-L) | Ormhoj et al., Clin. Cancer Res., (2019) (PMID: 31439577) |
| CD103 | M290 | Zhang et al., Am. J. Transplant., 9(9):2012-23 (2009) |
| | | (PMID: 19645708) |
| FAP | FAP5 | Wang et al., Cancer Immunol. Res., 2(2):154-66 (2014) |
| | | (PMID: 24778279) |
| CD70 | Human CD70 | Park et al., Oral Oncol., 78:145-150 (2018) (PMID: 29496042) |
| MUC16 | 4H11 | Koneru et al., Oncoimmunol., 4(3):e994446 (2015) (PMID: 25949921) |
| IL13Ra2 | IL13R | Kong et al., Clin. Cancer Res., 18(21):5949-60 (2012) |
| | | (PMID: 22966020) |

In some cases, a viral particle described herein (e.g., a viral particle containing nucleic acid encoding a selected antigen receptor) can be used to target any appropriate type of cell. For example, a viral particle described herein can be used to target cancer cells presenting an antigen (e.g., a tumor antigen) associated with a particular cancer. Examples of antigens associated with a particular cancer include, without limitation, CD19 (associated with B cell lymphomas, acute lymphoblastic leukemia (ALL), and chronic lymphocytic leukemia (CLL)), AFP (associated with germ cell tumors and/or hepatocellular carcinoma), CEA (associated with bowel cancer, lung cancer, and/or breast cancer), CA-125 (associated with ovarian cancer), MUC-1 (associated with breast cancer), ETA (associated with breast cancer), MAGE (associated with malignant melanoma), CD33, CD123, CLEC12A (myeloid diseases), CD22, CD20, kappa, lambda (B cell malignancies), folate receptor alpha (ovarian cancer), BCMA, CS-1, CD138, CD38 (multiple myeloma), universal peptide receptor, T cell receptor (T cell lymphoma and leukemia), CD5, CD7, and CD2 (T cell lymphoma and leukemia). For example, a viral particle described herein can be used to target disease cells (e.g., disease cells other than cancer cells) presenting an antigen (e.g., a disease-associated antigen) associated with a particular disease (e.g., a disease other than cancer). Examples of antigens with a particular disease include, without limitation, Viral Capsid Antigen (associated with Mononucleosis) and GP120 (associated with HIV).

A viral particle described herein (e.g., a viral particle containing nucleic acid encoding a selected antigen receptor) can be engineered to contain nucleic acid encoding a selected antigen receptor using any appropriate methods and/or techniques. In some cases, nucleic acid encoding a selected antigen receptor can be incorporated into a viral genome. Nucleic acid encoding a selected antigen receptor can be introduced into a viral particle using any appropriate methods and/or techniques. For example, nucleic acid encoding a selected antigen receptor can be introduced into a viral particle using molecular cloning techniques. In some cases, a viral particle described herein can contain nucleic acid encoding a CAR targeting CD19 (e.g., an anti-CD19 CAR). An exemplary nucleic acid sequence encoding an anti-CD19 CAR can include a sequence as set forth in SEQ ID NO:1.

In some cases, a viral particle described herein (e.g., a viral particle containing nucleic acid encoding a selected antigen receptor) can target (e.g., can be engineered to target) one or more T cells. For example, a viral particle described herein can include one or more exogenous molecules that can direct the viral particle to target (e.g., target and infect) one or more T cells in vivo. Exogenous molecules that can direct a viral particle to target one or more T cells also can be referred to as targeting molecules. An exogenous molecule can target any appropriate type of T cell. In some cases, a T cell can be a naïve T cell. In some cases, a T cell can be an immunosuppressive T cell. Examples of T cells that can be targeted by one or more viral particles described herein include, without limitation, cytotoxic T cells (CTLs; e.g., CD4⁺ CTLs and/or CD8⁺ CTLs), regulatory T cells, and memory subsets of T cells. In some cases, a viral particle described herein can target a particular type of T cell. An exogenous molecule can target any appropriate molecule present on one or more T cells. Examples of molecules present on a T cell that can be targeted via a targeting molecule incorporated into a viral particle described herein include, without limitation, a component of a T cell receptor (such as a CD3 polypeptide, TCR-α chain polypeptide, a TCR-β chain polypeptide, or a ζ-chain polypeptide), CD2, CD8, CD4, Cd25, CD5, and CD7. An exogenous molecule can be any appropriate type of molecule that can target a molecule present on one or more T cells. In some cases, an exogenous molecule can contain an antigen-binding domain (e.g., an antigen-binding domain that recognizes an antigen on T cell) such as single-chain variable fragments (scFvs), antibodies, antigen-binding fragments (Fabs), single-domain antibodies (sdAbs), affibodies, and/or receptor proteins. In some cases, a targeting molecule incorporated into a viral particle described herein can be an antigen (e.g., an antigen that is recognized by an antigen-binding domain on a T cell). In some cases, an exogenous molecule can be a receptor (e.g., a receptor that forms a complex with a ligand on T cell). In some cases, a targeting molecule can be a ligand (e.g., a ligand that forms a complex with a receptor on a T cell). Examples of targeting molecules that can target a viral particle described herein to one or more T cells include, without limitation, anti-CD3 molecules containing CD3 antigen-binding domains (e.g., anti-CD3 scFvs), CD2, CD8, CD4, Cd25, CD5, and CD7.

In cases where a viral particle described herein (e.g., a viral particle containing nucleic acid encoding a selected antigen receptor) includes one or more exogenous molecules (e.g., one or more targeting molecules that can direct the viral particle to target one or more T cells), the targeting molecule can be engineered into the viral particle as an exogenous component of the viral particle using any appropriate methods and/or techniques. For example, a viral particle described herein can be engineered to include one or more viral components (e.g., modified genetic material (e.g., a viral genome), a modified capsid, and/or a modified envelope) that are modified to include one or more targeting molecules. In some cases, a viral particle described herein can include a modified envelope that includes one or more targeting molecules. An exogenous targeting molecule can be incorporated into a viral component (e.g., an envelope) using any appropriate methods and/or techniques. For example, a viral particle can include nucleic acid encoding a targeting molecule such that the targeting molecule is expressed and incorporated into a component (e.g., an envelope) of the viral particle. In cases where a viral particle described herein includes one or more targeting molecules containing a CD3 antigen-binding domain (e.g., one or more anti-CD3 scFvs), the viral particle can contain nucleic acid encoding the targeting molecule(s). For example, a viral particle including one or more anti-CD3 scFvs can contain nucleic acid encoding the anti-CD3 scFvs. An exemplary nucleic acid sequence encoding an anti-CD3 scFv can include a sequence as set forth in SEQ ID NO:2.

In some cases, a viral particle described herein (e.g., a viral particle containing nucleic acid encoding a selected antigen receptor) can include a lentiviral backbone and a measles virus envelope, can contain nucleic acid encoding an antigen receptor (e.g., a CAR) that can target CD19 (e.g., an anti-CD19 CAR), and can contain nucleic acid encoding a targeting molecule (e.g., an antigen-binding domain that recognizes an antigen on T cell such as an anti-CD3 scFv) such that the measles virus envelope presents the antigen-binding domain that recognizes an antigen on T cell. For example, a viral particle described herein can have a lentiviral backbone and a measles virus envelope, can contain nucleic acid encoding a CAR targeting CD19 as set forth in SEQ ID NO:1, and can contain nucleic acid encoding an anti-CD3 scFv as set forth in SEQ ID NO:2, such that the measles virus envelope presents the anti-CD3 scFv.

This document also provides cells capable of producing one or more viral particles described herein (e.g., one or more viral particles containing nucleic acid encoding a selected antigen receptor). A cell capable of producing one or more viral particles described herein can be any appropriate type of cell. Examples of cells that can be used to produce one or more viral particles described herein include, without limitation, 2293T, HEK293F, and Sup Tl cells. Any appropriate method and/or technique can be used to engineer a cell capable of producing one or more viral particles described herein (e.g., one or more viral particles containing nucleic acid encoding a selected antigen receptor). For example, a cell can be transduced with one or more viral particles described herein to engineer the cell to be capable of producing one or more viral particles described herein. In some cases, a cell can be transduced with one or more viral particles containing nucleic acid encoding a selected antigen receptor (e.g., nucleic acid encoding a CAR targeting CD19 as set forth in SEQ ID NO: 1), and containing nucleic acid sequence encoding a targeting molecule that can direct the viral particle to target one or more T cells (e.g., nucleic acid encoding an anti-CD3 scFv as set forth in SEQ ID NO:2) to engineer the cell to be capable of producing one or more viral particles described herein. To generate these cells, virus producing cells can be transfected with a CAR plasmid, an envelope plasmid, and a packaging plasmid. Cells can then be purified and injected in situ or in vivo.

In some cases, a cell capable of producing one or more viral particles described herein (e.g., one or more viral particles containing nucleic acid encoding a selected antigen receptor) can produce one or more viral particles including a lentiviral backbone and a measles virus envelope, where the viral particle(s) contain nucleic acid encoding an antigen receptor (e.g., a CAR) that can target CD19 (e.g., an anti-CD19 CAR), and where the viral particle(s) contain nucleic acid encoding an antigen-binding domain that recognizes an antigen on T cell (e.g., an anti-CD3 scFv) such that the measles virus envelope presents the antigen-binding domain that recognizes an antigen on T cell. For example, a cell capable of producing one or more viral particles described herein can produce one or more viral particles having a lentiviral backbone and a measles virus envelope, where the viral particle(s) contain nucleic acid encoding a CAR targeting CD19 as set forth in SEQ ID NO:1, and where the viral particle(s) contain nucleic acid encoding an anti-CD3 scFv as set forth in SEQ ID NO:2, such that the measles virus envelope presents the anti-CD3 scFv.

This document also provides methods and materials for generating T cells expressing the selected antigen receptor within the mammal. In some cases, one or more viral particles provided herein (e.g., one or more viral particles containing nucleic acid encoding a selected antigen receptor) and/or one or more cells capable of producing such viral particles can be administered to a mammal to generate T cells expressing that selected antigen receptor within the mammal. For example, one or more viral particles containing nucleic acid encoding a selected antigen receptor (e.g., a selected CAR) administered to a mammal can transduce one or more T cells within the mammal (e.g., to introduce the nucleic acid encoding the selected antigen receptor into the T cells such that the T cells express the selected antigen receptor) to generate one or more T cells expressing the selected antigen receptor (e.g., one or more CART cells) within the mammal. For example, one or more cells capable of producing viral particles containing nucleic acid encoding a selected antigen receptor (e.g., a selected CAR) administered to a mammal can result in the production of infectious viral particles, which in turn can transduce one or more T cells within the mammal (e.g., to introduce the nucleic acid encoding the selected antigen receptor into the T cells such that the T cells express the a selected antigen receptor) to generate one or more T cells expressing the selected antigen receptor (e.g., one or more CART cells) within the mammal.

In some cases, when one or more viral particles provided herein (e.g., one or more viral particles containing nucleic acid encoding a selected antigen receptor) and/or one or more cells capable of producing such viral particles are administered to a mammal to generate T cells expressing that selected antigen receptor within the mammal, the T cells expressing that selected antigen receptor do not target normal (e.g., healthy) cells.

In some cases, when one or more viral particles provided herein (e.g., one or more viral particles containing nucleic acid encoding a selected antigen receptor) and/or one or more cells capable of producing such viral particles are administered to a mammal to generate T cells expressing that selected antigen receptor within the mammal, the T cells expressing that selected antigen receptor can avoid the mammal's immune system.

In some cases, when one or more viral particles provided herein (e.g., one or more viral particles containing nucleic acid encoding a selected antigen receptor) and/or one or more cells capable of producing such viral particles are administered to a mammal to generate T cells expressing that selected antigen receptor within the mammal, the T cells expressing that selected antigen receptor can target non cancer cells (e.g., auto immune disease cells) and can be non-pathogenic.

In some cases, when one or more cells capable of producing one or more viral particles provided herein (e.g., one or more viral particles containing nucleic acid encoding a selected antigen receptor) are administered to a mammal to generate T cells expressing that selected antigen receptor within the mammal, the one or more cells capable of producing one or more viral particles provided herein can continue to generate viral particles in situ. In some cases, these cells can produce virus for about 2 to 3 days until they are eliminated.

One or more viral particles provided herein (e.g., one or more viral particles containing nucleic acid encoding a selected antigen receptor) and/or one or more cells capable of producing such viral particles can be administered to any appropriate mammal to generate T cells expressing that selected antigen receptor within the mammal. In some cases, a mammal (e.g., a human) can have cancer. In some cases, a mammal (e.g., a human) can have a disease other than cancer. Examples of mammals within which T cells expressing a selected antigen receptor can be generated as described herein include, without limitation, humans, non-human primates (e.g., monkeys), dogs, cats, horses, cows, pigs, sheep, mice, and rats. In some cases, one or more viral particles provided herein and/or one or more cells capable of producing such viral particles can be administered to a human to generate T cells expressing a selected antigen receptor within the human.

In some cases, one or more viral particles provided herein (e.g., one or more viral particles containing nucleic acid encoding a selected antigen receptor) and/or one or more cells capable of producing such viral particles can be administered to a mammal having cancer to treat the mammal. For example, one or more viral particles containing nucleic acid encoding a selected tumor antigen receptor and/or one or more cells capable of producing viral particles containing nucleic acid encoding a selected tumor antigen receptor can be administered to a mammal having a cancer (e.g., a cancer containing cancer cells presenting a tumor antigen targeted by the selected tumor antigen receptor) to generate T cells expressing the selected tumor antigen receptor within the mammal to treat the mammal (e.g., by specifically targeting the cancer cells presenting a tumor antigen targeted by the selected tumor antigen receptor).

In some cases, when treating a mammal having cancer as described herein (e.g., by administering one or more viral particles containing nucleic acid encoding a selected antigen receptor and/or one or more cells capable of producing such viral particles to a mammal to generate T cells expressing a selected antigen receptor within the mammal), the mammal can have any type of cancer. A cancer can be a primary cancer or a metastatic cancer. In some cases, a cancer can include one or more solid tumors. In some cases, a cancer can be a cancer in remission. In some cases, a cancer can include quiescent (e.g., dormant or non-dividing) cancer cells. In some cases, a cancer can be cancer that has escaped chemotherapy and/or has been non-responsive to chemotherapy. Examples of cancers that can be treated by administering one or more viral particles containing nucleic acid encoding a selected antigen receptor and/or one or more cells capable of producing such viral particles to a mammal to generate T cells expressing an antigen receptor within the mammal include, without limitation, B cell lymphomas, ALLs, CLLs, germ cell tumors, hepatocellular carcinomas, bowel cancers, lung cancers, breast cancers, ovarian cancers, malignant melanomas, AML, multiple myeloma, thyroid cancer, head and neck cancers, and brain cancers. In some cases, a mammal treated as described herein can have a cancer containing cancer cells presenting a tumor antigen. Examples of tumor antigens associated with one or more particular cancers include, without limitation, CD19 (associated with B cell lymphomas, ALLs, and CLLs), AFP (associated with germ cell tumors and/or hepatocellular carcinomas), CEA (associated with bowel cancers, lung cancers, and/or breast cancers), CA-125 (associated with ovarian cancers), MUC-1 (associated with breast cancers), ETA (associated with breast cancers), MAGE (associated with malignant melanomas), CD38 (associated with multiple myeloma), CD33, CD123, CLEC12A (myeloid diseases), CD22, CD20, kappa, lambda (B cell malignancies), folate receptor alpha (ovarian cancer), BCMA, CS-1, CD138, CD38 (multiple myeloma), universal peptide receptor, T cell receptor (T cell lymphoma and leukemia), CD5, CD7, and CD2 (T cell lymphoma and leukemia). In some cases, a human having a B cell lymphomas, ALL, or CLL, can be treated by administering one or more viral particles expressing a CAR targeting CD19 to generate CART cells expressing a CAR targeting CD19 within the human.

In some cases, when treating a mammal having cancer as described herein (e.g., by administering one or more viral particles containing nucleic acid encoding a selected antigen receptor and/or one or more cells capable of producing such viral particles to a mammal to generate T cells expressing a selected antigen receptor within the mammal), the mammal can be identified as having cancer. Any appropriate method can be used to identify a mammal as having cancer. For example, imaging techniques can be used to identify a mammal as having cancer.

In some cases, when treating a mammal having cancer as described herein (e.g., by administering one or more viral particles containing nucleic acid encoding a selected antigen receptor and/or one or more cells capable of producing such viral particles to a mammal to generate T cells expressing a selected antigen receptor within the mammal), the treatment can reduce one or more symptoms of the cancer in the mammal. For example, the treatment can reduce the number of cancer cells (e.g., cancer cells expressing a tumor antigen) within a mammal. For example, the treatment can reduce the size (e.g., volume) of one or more tumors (e.g., tumors expressing a tumor antigen) within a mammal.

In some cases, one or more viral particles provided herein (e.g., one or more viral particles containing nucleic acid encoding a selected antigen receptor) and/or one or more cells capable of producing such viral particles can be administered to a mammal having a disease (e.g., a disease other than cancer) to treat the mammal. For example, one or more viral particles containing nucleic acid encoding a selected disease-associated antigen receptor and/or one or more cells capable of producing viral particles containing nucleic acid encoding a selected disease-associated antigen receptor can be administered to a mammal having a disease (e.g., a disease containing disease cells presenting a disease-associated antigen targeted by the selected disease-associated antigen receptor) to generate T cells expressing a selected antigen receptor within the mammal to treat the mammal (e.g., by specifically targeting the disease cells presenting a disease-associated antigen targeted by the selected disease-associated antigen receptor).

In some cases, when treating a mammal having a disease (e.g., a disease other than cancer) as described herein (e.g., by administering one or more viral particles containing nucleic acid encoding a selected antigen receptor and/or one or more cells capable of producing such viral particles to a mammal to generate T cells expressing a selected antigen receptor within the mammal), the mammal can have any type of disease. For example, a mammal having an infection (e.g., a viral infection or a bacterial infection) can be treated as described herein. Examples of infections that can be treated as described herein include, without limitation, influenza infections, hepatitis infections, fungal infections, CMV infections, EBV associated infections, HHV6 infections, and viral infections. In some cases, an infected cell can present a disease-associated antigen (e.g., a viral antigen or a bacterial antigen). Examples of disease-associated antigens associated with one or more particular infections include, without limitation, glactomannan (associated with aspergillus and fungal infections) and candin (candida antigen).

In some cases, when treating a mammal have a disease (e.g., a disease other than cancer) as described herein (e.g., by administering one or more viral particles containing nucleic acid encoding a selected antigen receptor and/or one or more cells capable of producing such viral particles to a mammal having a disease other than cancer to generate T cells expressing a selected antigen receptor within the mammal), the mammal can be identified as having a disease. Any appropriate method can be used to identify a mammal as having a disease. For example, when a disease is an infection, an infestation, and/or an autoimmune disease can be used to identify a mammal as having an infection.

In some cases, when treating a mammal having a disease (e.g., a disease other than cancer) as described herein (e.g., by administering one or more viral particles containing nucleic acid encoding a selected antigen receptor and/or one or more cells capable of producing such viral particles to a mammal to generate T cells expressing a selected antigen receptor within the mammal), the treatment can reduce one or more symptoms of the disease in the mammal. For example, when the disease is an infection, the treatment can reduce the number of infected cells within a mammal.

One or more viral particles provided herein (e.g., one or more viral particles containing nucleic acid encoding a selected antigen receptor) and/or one or more cells capable of producing such viral particles can be administered to a mammal to generate T cells expressing that selected antigen receptor within the mammal using any appropriate method and/or technique. In some cases, one or more viral particles described herein can be administered into or near a tumor. Examples of methods of administering one or more viral particles described herein to a mammal can include, without limitation, injections such as intratumoral, intravenous (IV), intradermal (ID), subcutaneous, in situ in the lymph node, or intra peritoneal injections. In some cases, when one or more viral particles described herein are administered to a mammal as described herein, the administration can be IV administration or intratumoral administration. In some cases, when one or more cells capable of producing one or more viral particles described herein are administered to a mammal as described herein, the administration can be intratumoral administration.

One or more T cells expressing that selected antigen receptor generated within a mammal as described herein (e.g., by administering one or more viral particles containing nucleic acid encoding a selected antigen receptor and/or one or more cells capable of producing such viral particles to a mammal to generate T cells expressing a selected antigen receptor within the mammal) can target a cell (e.g., a cell presenting an antigen recognized by the selected antigen receptor) in any appropriate location within the mammal. In some cases, a T cell expressing a selected antigen generated within a mammal as described herein can target a cell at a primary location (e.g., a site of administration) within the mammal. In some cases, a T cell expressing a selected antigen generated within a mammal as described herein can target a cell at one or more secondary locations (e.g., a site different from the site of administration) within a mammal. In some cases, a T cell expressing a selected antigen generated within a mammal as described herein can target cells at both a primary location and at one or more secondary locations within a mammal having cancer. For example, a T cell expressing a selected antigen generated within a mammal as described herein can target cells at a primary location within a mammal and can leave the primary location and can migrate to target cells at one or more secondary locations.

In some cases, one or more viral particles provided herein (e.g., one or more viral particles containing nucleic acid encoding a selected antigen receptor) and/or one or more cells capable of producing such viral particles can be administered to a mammal together with one or more additional therapeutic agents. For example, when treating a mammal having cancer as described herein, one or more viral particles provided herein and/or one or more cells capable of producing such viral particles can be administered to a mammal together with one or more additional anti-cancer treatments. Examples of anti-cancer treatments include, without limitation, surgery, radiation therapy, chemotherapy, targeted therapy, hormonal therapy, angiogenesis inhibitors, immunosuppressants, immunostimulants, immunomodulatory agents, and small molecule inhibitors. For example, when treating a mammal having an infection as described herein, one or more viral particles provided herein and/or one or more cells capable of producing such viral particles can be administered to a mammal together with one or more additional agents used to treat an infection. Examples of agents used to treat infections include, without limitation, antivirals, antibiotics, and antifungals. In cases where one or more viral particles provided herein and/or one or more cells capable of producing such viral particles are used with additional therapeutic agents, the one or more additional therapeutic agents can be administered at the same time or independently. In some cases, one or more viral particles provided herein and/or one or more cells capable of producing such viral particles can be administered first, and the one or more additional therapeutic agents administered second, or vice versa.

In some cases, a T cell generated as described herein to express a selected antigen receptor using one or more viral particles provided herein (and/or one or more cells capable of producing such viral particles) can express the selected antigen receptor (an antigen receptor heterologous to that T cell) in addition to a native antigen receptor of that T cell (e.g., the T cell's native TCR).

In some cases, virus producing cells described herein can be 293T cells or any other type of virus producing cell. In some cases, such virus producing cells can be injected intravenously, intra tumorally, subcutaneously, or regionally into local lymph nodes. As described herein, a virus producing cell can be transfected with a lentivirus or retrovirus plasmid encoding a CAR construct (e.g., stably transfected with a CAR-encoding sequence, envelope-encoding sequence, and packaging plasmid(s)) and cryopreserved for future use. In some cases, virus producing cells described herein can be transfected with a lentivirus or retrovirus plasmid encoding a CAR construct with engineered envelops directed to T cells. Examples of such envelop polypeptides include, without limitation, envelops engineered to include an anti-CD3, anti-CD2, anti-CD5, anti-CD4, anti-CD8 antibody (e.g., an anti-CD3, anti-CD2, anti-CD5, anti-CD4, or anti-CD8 scFv antibody) or other T cell molecule directed antibody to allow targeted and specific transduction of T cells.

In some cases, a CAR construct can be a first, second, third, or subsequent generation CAR. In some cases, a CAR construct can be designed to promote CD3 zeta stimulation and can include one or more domains of CD28, 41BB, CD27, CD2, OX40, ICOS, other T cell stimulatory molecules, and/or members of the TNFα receptor family. As described herein, a CAR can be directed to any appropriate antigen present on a tumor cell such as those set forth in Table 1.

The invention will be further described in the following examples, which do not limit the scope of the invention described in the claims.

### EXAMPLES

### Example 1: In Vivo Stimulation and Generation of CART Cells Using Engineered Lentivirus

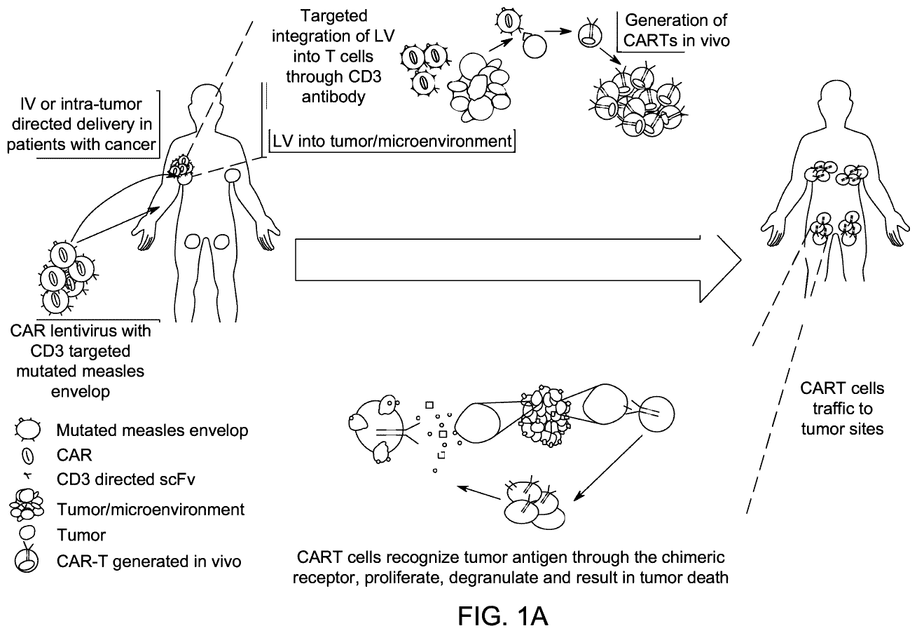

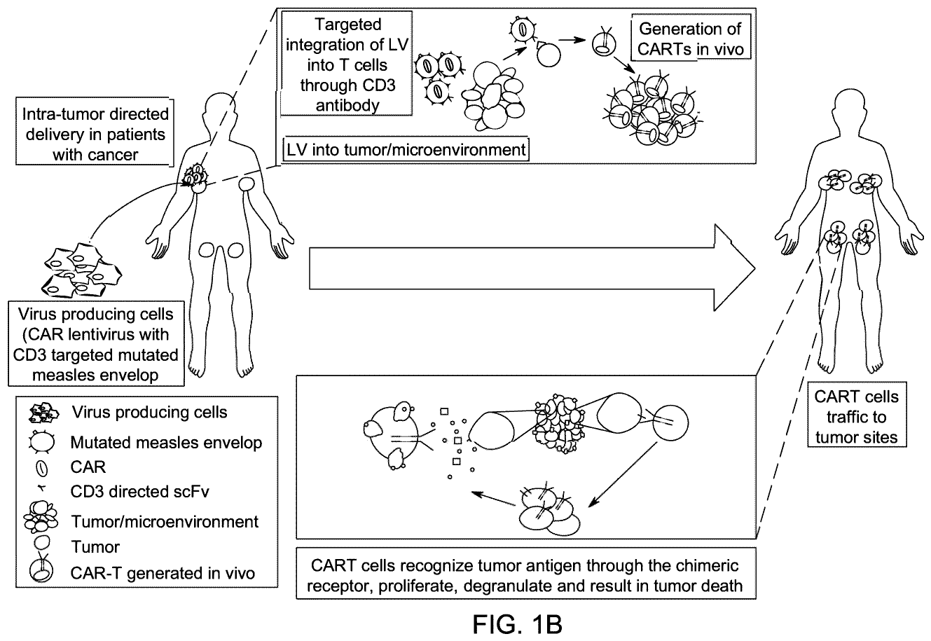

A platform was developed to generate CART cells in vivo. In this platform, lentiviruses are injected intravenously or in situ (e.g., into a tumor site). These virus particles attract T cells and stimulate and transduce the T cells to generate CART cells in situ/in vivo. Specifically, the lentiviruses encode a CD3 targeted envelope and a CAR plasmid to specifically transduce and stimulate T cells in situ and to generate CART cells in situ. See, e.g., FIG. 1A.

### Methods/Experimental Design

### Experimental Design on Human T Cells

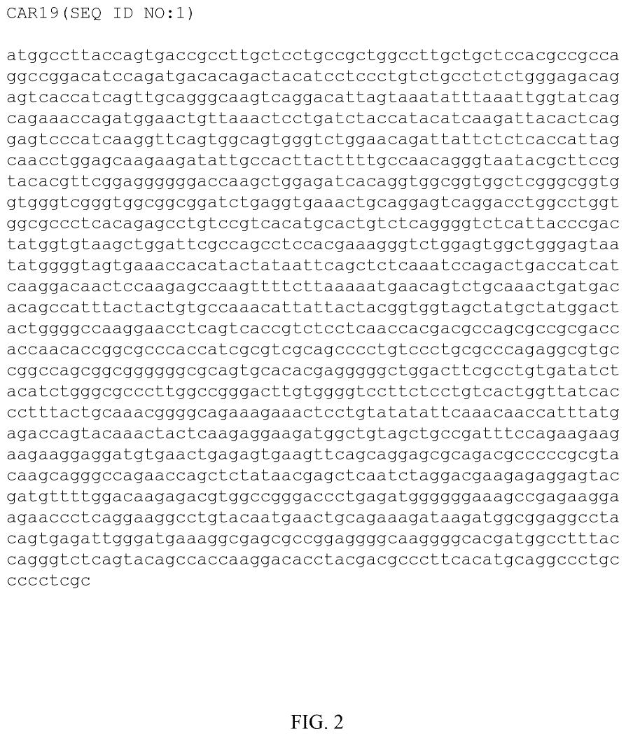

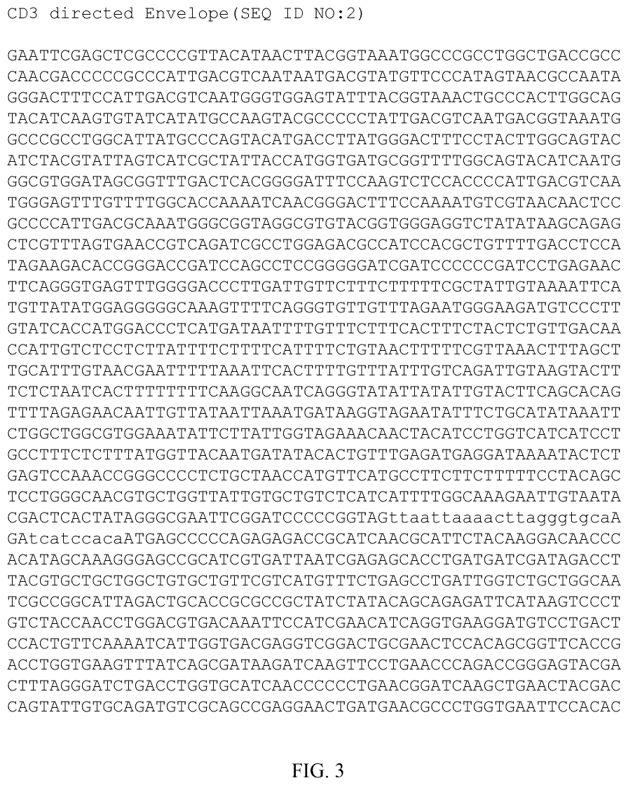

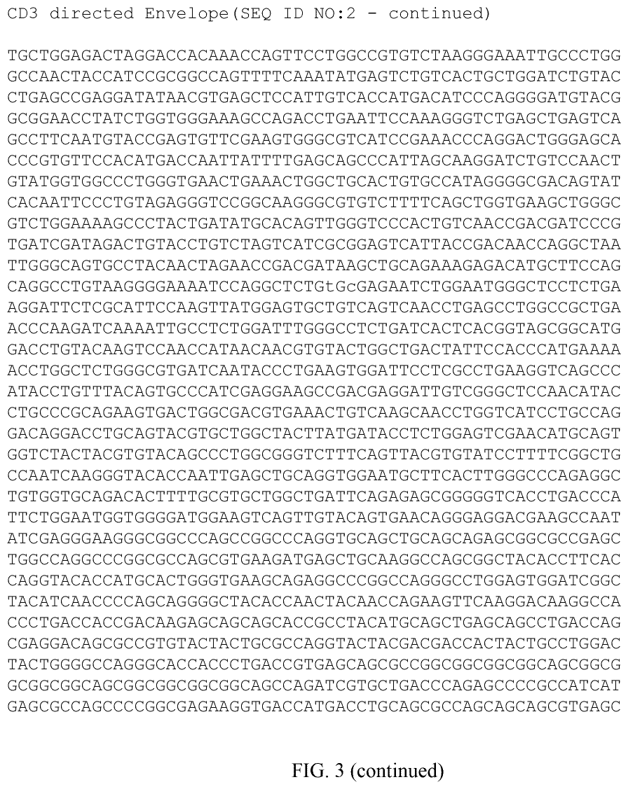

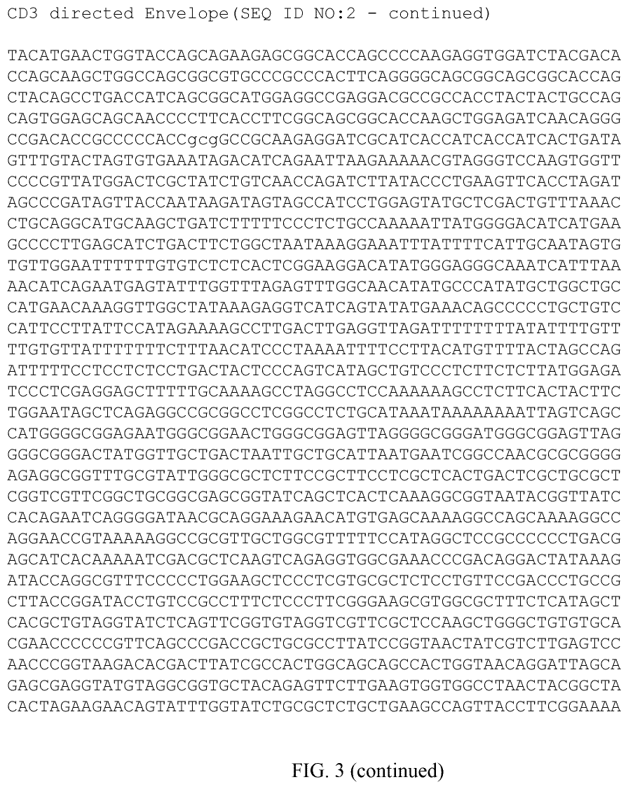

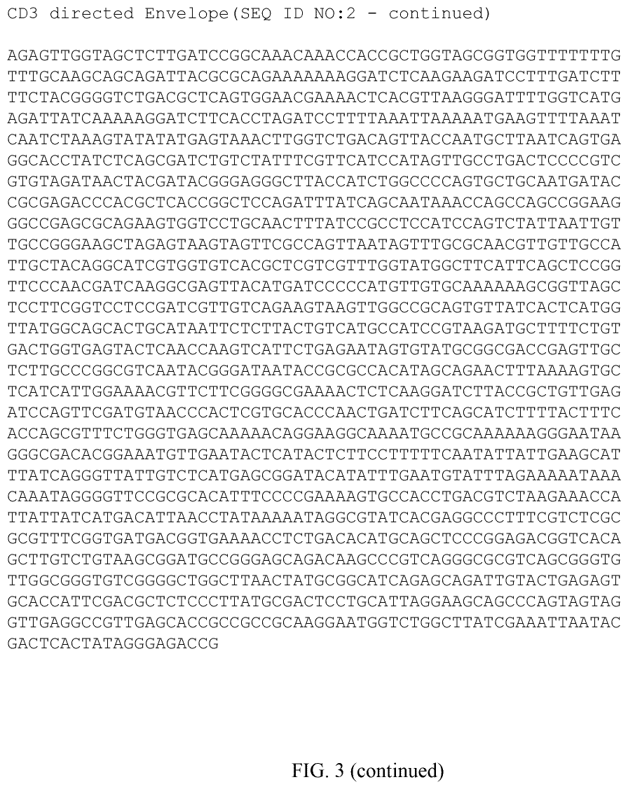

This platform is applied to human T cells in mouse models. Two plasmids are generated: 1) a CD3 directed, mutated measles envelope plasmid (FIG. 2), and 2) a CAR lentivirus plasmid (FIG. 3). CAR19 in B cell leukemia xenografts is used. These plasmids are used for packaging and lentivirus production (as described below).

Two mouse models are employed:
1. Xenograft models: NSG mice are engrafted with the CD19 positive, luciferase positive cell line NALM6 subcutaneously and then engraftment is confirmed by bioluminescence imaging. Mice are then treated with human PBMCs intravenously and intra-tumor injection of our lentivirus particles. Generation of CART cell is measured by flow cytometry. Trafficking of CARTs to tumor sites is assessed and anti-tumor response is measured by bioluminescence imaging as a measure of disease burden.
2. Humanized Immune System (HIS) mice from the Jackson Laboratory: HIS mice are injected with fetal CD34+ cells as neonates and therefore develop human hematopoiesis. Mice are engrafted with the CD19+ cell line NALM6, as previously used. Similarly, CART19 are generated in vivo through the intratumoral injection of lentivirus particles. The activity of CART19 cells in eradication of NALM6 is measured and compared that to ex vivo generated lenti-virally transduced CART 19 cells (e.g., as currently used in the clinic).

### Generation of CAR Plasmid:

The anti-CD19 clone FMC63 is de novo synthesized into a CAR backbone using 41BB and CD3 zeta and is then cloned into a third generation lentivirus backbone.

To generate control CART19 cells, normal donor T cells are negatively selected using pan T cell kit and expanded ex vivo using anti-CD3/CD28 Dynabeads (Invitrogen, added on the first day of culture). T cells are transduced with lentiviral supernatant one day following stimulation at a multiplicity of infection (MOI) of 3. The anti-CD3/CD28 Dynabeads are removed on day 6 and T cells are grown in T-cell media (X-vivo 15 media, human serum 5%, penicillin, streptomycin and glutamax) for up to 15 days and then cryopreserved for future experiments. Prior to all experiments, T cells are thawed and rested overnight at 37° C.

### Cells

The NALM6 cell line is obtained from the ATCC and maintained in R10 media (RPMI media, 10% fetal calf serum, penicillin, and streptomycin). NALM6-cells transduced with luciferase-GFP cells under the control of the EF1α promoter are used in some experiments as indicated. De-identified primary human acute lymphoblastic leukemia (ALL) specimens are obtained. All samples are obtained after informed, written consent. For all functional studies, cells are thawed at least 12 hours before analysis and rested overnight at 37° C.

### Flow Cytometry Analysis

Anti-human antibodies are purchased from Biolegend, eBioscience, or BD Biosciences. Cells are isolated from in vitro culture or from animals, washed once in PBS supplemented with 2% fetal calf serum, and stained at 4° C. after blockade of Fc receptors. For cell number quantitation, Countbright beads (Invitrogen) are used according to the manufacturer's instructions (Invitrogen). In all analyses, the population of interest is gated based on forward vs. side scatter characteristics followed by singlet gating, and live cells are gated using Live Dead Aqua (Invitrogen). Surface expression of anti-CD19 CAR is detected by staining with an Alexa Fluor 647-conjugated goat anti-mouse F (ab′) 2 antibody from Jackson Immunoresearch.

### T Cell Function Assays:

### 1. T Cell Degranulation and Intracellular Cytokine Assays

Briefly, T cells are incubated with target cells at a 1:5 ratio. After staining for CAR expression; CD107a, CD28, CD49d, and monensin are added at the time of incubation. After 4 hours, cells are harvested and stained for CAR expression, CD3 and Live Dead staining (Invitrogen). Cells are fixed and permeabilized (FIX & PERM® Cell Fixation & Cell Permeabilization Kit, Life technologies) and intracellular cytokine staining is then performed.

### 2. Proliferation Assays:

T cells are washed and resuspended at 1×10⁷/mL in 100 μL of PBS and labeled with 100 μL of CFSE 2.5 μM (Life Technologies) for 5 minutes at 37° C. The reaction is then quenched with cold R10, and the cells are washed three times. Targets are irradiated at a dose of 100 Gy. T cells are incubated at a 1:1 ratio with irradiated target cells for 120 hours. Cells are then harvested, stained for CD3, CAR and Live Dead aqua (Invitrogen), and Countbright beads (Invitrogen) are added prior to flow cytometric analysis

### 3. Cytotoxicity Assays:

NALM6-Luc cells or CFSE (Invitrogen) labelled primary ALL samples are used for cytotoxicity assay. In brief, targets are incubated at the indicated ratios with effector T cells for 4 or 16 hours. Killing is calculated either by bioluminescence imaging on a Xenogen IVIS-200 Spectrum camera or by flow cytometry. For the latter, cells are harvested; Countbright beads and 7-AAD (Invitrogen) are added prior to analysis. Residual live target cells are CFSE+ 7-AAD−.

### 4. Secreted Cytokine Measurement:

Effector and target cells are incubated at a 1:1 ratio in T cell media for 24 or 72 hours as indicated. Supernatant is harvested and analyzed by 30-plex Luminex array according to the manufacturer's protocol (Invitrogen).

### Results

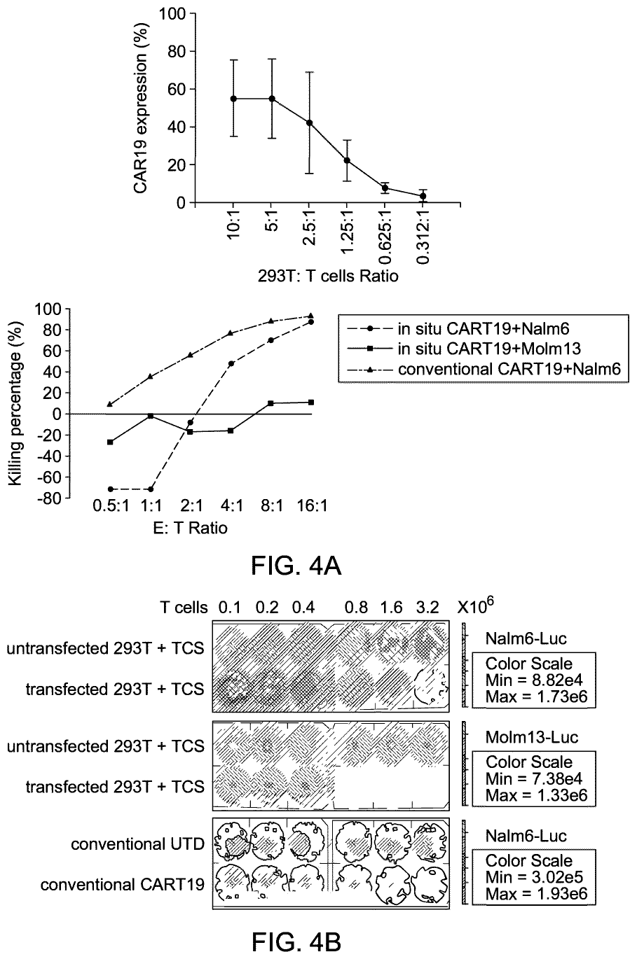

Virus producing cells were capable of transducing T cells and generating CART cells. Here, 293 T cells were transfected with a CAR plasmid, lipofectamine 3000, and packaging plasmids, in the presence of T cells. This resulted in the generation of CART cells. The optimal ratio in 5:1 virus producing cell: T cell resulted in 50-60% transduction efficiency (FIG. 4A). Virus producing cells generated CART19 cells exhibiting potent anti-tumor efficacy against CD19⁺ tumors (FIG. 4B).

### Example 2: In Vivo Generation of CART Cells Through the Use of CD3 Targeted Lentivirus Producing Cells

A platform was developed to generate CART cells in vivo. In this platform, lentivirus producing cells are injected in situ (e.g., into a tumor site). These cells are transduced with viral particles described in Example 1 to produce virus particles that attract T cells, and stimulate and transduce the T cells to generate CART cells in situ/in vivo. Specifically, the lentiviruses encode a CD3 targeted envelope and a CAR plasmid to specifically transduce and stimulate T cells in situ, and generate CART cells in situ. See, e.g., FIG. 1B.

### Example 3: Generating CART Cells In Vivo Through the Use of Lentivirus Producing Cells

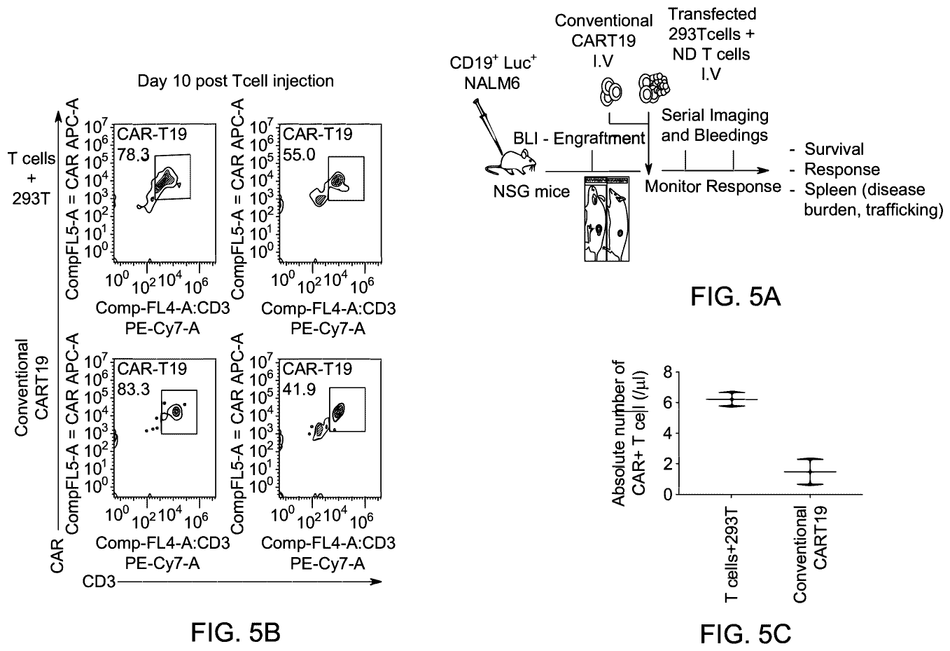

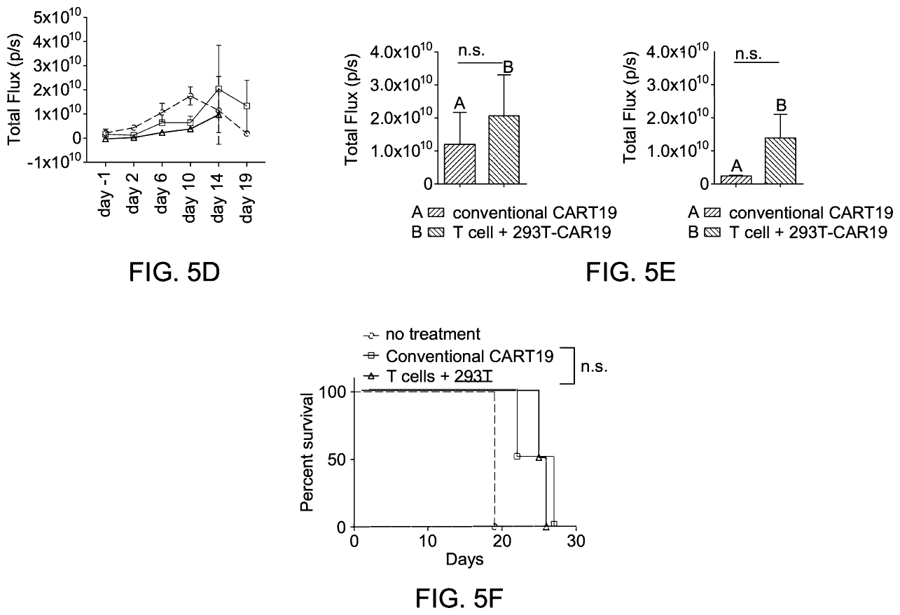

The following was performed. Immunocompromised mice (male and female 8-12 week old NOD-SCID-IL2rγ^{-/-} (NSG) mice) were engrafted with the human CD19⁺ luciferase⁺ tumor cell line NALM6 subcutaneously. Engraftment was confirmed by bioluminescence imaging, and then mice were randomized to be treated with either conventional ex vivo generated CART19 or with 293T cells transfected with CAR plasmid followed by stimulated T cells (FIG. 5A). Ten days later, the mice were bled to look for CART cell generation. The mice treated with transfected 293T cells followed by human T cells had CART19 generated in vivo, and at higher concentration compared to ex vivo generated CART19 (FIG. 5B-C). Follow up of these mice showed similar anti-tumor activity and overall survival that is improved compared to untreated mice (FIG. 5D-F).

### Cells Line

NALM6 was purchased from ATCC, (Manassas, VA, USA). These cells were transduced with a luciferase-ZsGreen lentivirus (Addgene, Cambridge, MA, USA) and sorted to 100% purity. Cell lines were cultured in R10 made with RPMI 1640 (Gibco, Gaithersburg, MD, USA), 10% FBS (Millipore Sigma, Ontario, Canada), and 1% Penicillin-Streptomycin-Glutamine (Gibco, Gaithersburg, MD, USA).

### Ex Vivo Generated CART Cells

Peripheral blood mononuclear cells (PBMC) were isolated from de-identified donor blood apheresis cones using SepMate tubes (STEMCELL Technologies, Vancouver, Canada). T cells were separated with negative selection magnetic beads using EasySep™ Human T Cell Isolation Kit (STEMCELL Technologies, Vancouver, Canada), and monocytes were positively selected using CD14⁺ magnetic beads (Miltenyi Biotec, Bergisch Gladbach, Germany). Primary cells were cultured in T Cell Media made with X-Vivo 15 (Lonza, Walkersville, MD, USA) with 10% human serum albumin (Corning Inc., Corning, NY, USA) and 1% Penicillin-Streptomycin-Glutamine (Gibco, Gaithersburg, MD, USA). CD19-directed CART cells were generated through the lentiviral transduction of normal donor T cells as described below. Second generation CAR 19 constructs were de novo synthesized (IDT) and cloned into a third generation lentivirus under the control of EF-la promotor. The CD19-directed single chain variable fragment was derived from the clone FMC63. A second generation 41BB co-stimulated (FMC63-41BBz) CAR construct was synthesized and used for these experiments. Lentiviral particles were generated through the transient transfection of plasmid into 293T virus producing cells in the presence of Lipofectamine 3000 (Invitrogen, Carlsbad, CA, USA), VSV-G, and packaging plasmids (Addgene, Cambridge, MA, USA). T cells isolated from normal donors were stimulated using Cell Therapy Systems Dynabeads CD3/CD28 (Life Technologies, Oslo, Norway) at 1:3 ratio and then transduced with lentivirus particles 24 hours after stimulation at a multiplicity of infection of 3.0. Magnetic bead removal was performed on Day 6, and CART cells were harvested and cryopreserved on Day 8 for future experiments. CART cells were thawed and rested in T cell medium 12 hours prior to their use.

### In Vivo Generated CART Cells

To generate CART cells in vivo, mice were treated with (1) the lentivirus producing cells where the lentivirus was capable of expressing the CAR plasmid and then (2) T cells at 5:1 ratio. T cells were activated ex vivo 24 hours prior to injection in mice. Peripheral blood sampling was performed 10 days later to determine the presence of CAR⁺ cells in the blood, and antitumor burden was monitored serially with bioluminescence imaging as a measure of residual disease.

### Example 4: Generating CART Cells In Vivo within Humans Through the Use of Lentivirus Producing Cells

The following is performed to generate CART cells in vivo using virus producing cells directly injected into a human, for example, intravenously or intra tumorally. To achieve this, human patients with cancer are first administered a treatment to stimulate their T cells systemically. Then, the human patients are administered virus producing cells that are designed to produce virus (e.g., lentivirus) capable of expressing nucleic acid encoding a CAR (a CAR lentivirus plasmid). The administration can be an intravenous, intratumoral, regional, local, systemic, or subcutaneous administration. Within the human, the virus producing cells are designed to produce the CAR virus in vivo, which in turn transduce T cells in vivo and generate CART cells in vivo. The in vivo generated CART cells can traffic to the tumor site, kill the tumor cells, produce cytokines, and exert their potent antitumor activity. The virus producing cells can serve as a local virus producing factory, producing virus transduced T cells and generating CART cells.

### Example 5: Stimulating T Cells In Vivo

Monoclonal antibodies directed to CD3, CD28, 41BB, ICOS, OX40, CD27, CD2, other T cell stimulating molecules, or a combination thereof (e.g., a combination of antibodies such as a combination of anti-CD3 and anti-CD28 antibodies) are used to stimulate T cells in vivo prior to, at the same time as, or after administering virus producing cells capable of producing virus having the ability to express a CAR after that virus infects, for example, a T cell. In some cases, biodegradable beads directed to CD3, CD28, 41BB, ICOS, OX40, CD27, CD2, other T cell stimulating molecules, or a combination thereof (e.g., beads containing a combination of antibodies such as a combination of anti-CD3 and anti-CD28 antibodies) are used to stimulate T cells in vivo prior to, at the same time as, or after administering virus producing cells capable of producing virus having the ability to express a CAR after that virus infects, for example, a T cell. In some cases, nanoparticles carrying antibodies to CD3, CD28, 41BB, ICOS, OX40, CD27, CD2, other T cell stimulating molecules, or a combination thereof (e.g., nanoparticles containing a combination of antibodies such as a combination of anti-CD3 and anti-CD28 antibodies) are used to stimulate T cells in vivo prior to, at the same time as, or after administering virus producing cells capable of producing virus having the ability to express a CAR after that virus infects, for example, a T cell. In some cases, the virus producing cells are engineered to produce viruses (e.g., a lentiviruses) designed to express an anti-CD3 antibody or other T cell stimulating molecule.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1-30. (canceled)

31. A method for generating T cells expressing a selected antigen receptor in vivo within a mammal, wherein said method comprises administering, to said mammal, a cell capable of producing a viral particle, wherein said viral particle comprises nucleic acid encoding said selected antigen receptor, wherein said viral particle is produced within said mammal and infects T cells comprising an endogenous T cell receptor within said mammal to form infected T cells, and wherein said infected T cells express said selected antigen receptor and said endogenous T cell receptor.

32. The method of claim 31, wherein said mammal is a human.

33. The method of claim 31, wherein said viral particle is a lentiviral particle.

34. The method of claim 31, wherein said antigen receptor is a chimeric antigen receptor.

35. The method of claim 31, wherein said viral particle can target one or more cancer cells.

36. The method of claim 31, wherein said one or more cancer cells express a tumor-specific antigen.

37. The method of claim 36, wherein said selected antigen receptor targets said tumor-specific antigen.

38. The method of claim 36, wherein said tumor-specific antigen is CD19.

39. The method of claim 38, wherein said antigen receptor targeting said tumor-specific antigen is a CAR targeting CD19.

40. The method of claim 39, wherein said nucleic acid encoding said CAR targeting CD19 comprises a nucleic acid sequence set forth in SEQ ID NO: 1.

41. The method of claim 31, wherein said exogenous molecule comprises an antigen-binding domain that can recognize CD3 and wherein said viral particle further comprises nucleic acid encoding said antigen-binding domain that can recognize CD3.

42. The method of claim 41, wherein said antigen-binding domain is a single-chain variable fragment.

43. The method of claim 42, wherein said nucleic acid encoding said single-chain variable fragment that can recognize CD3 comprises a nucleic acid sequence set forth in SEQ ID NO:2.

44. The method of claim 31, wherein said administration is intravenous injection or intratumoral injection.

45. The method of claim 31, wherein said nucleic acid encoding said selected antigen receptor comprises nucleic acid encoding a scFv set forth in Table 1.

46-52. (canceled)


SEQUENCE LISTING (2 sequences)

SEQ ID NO: 1 - DNA, 1458 bp, synthetic construct
  source 1..1458: mol_type = other DNA
atggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 2 - DNA, 7212 bp, synthetic construct
  source 1..7212: mol_type = other DNA
gaattcgagctcgccccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgggggatcgatccccccgatcctgagaacttcagggtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacaattgttataattaaatgataaggtagaatatttctgcatataaattctggctggcgtggaaatattcttattggtagaaacaactacatcctggtcatcatcctgcctttctctttatggttacaatgatatacactgtttgagatgaggataaaatactctgagtccaaaccgggcccctctgctaaccatgttcatgccttcttctttttcctacagctcctgggcaacgtgctggttattgtgctgtctcatcattttggcaaagaattgtaatacgactcactatagggcgaattcggatcccccggtagttaattaaaacttagggtgcaagatcatccacaatgagcccccagagagaccgcatcaacgcattctacaaggacaacccacatagcaaagggagccgcatcgtgattaatcgagagcacctgatgatcgatagaccttacgtgctgctggctgtgctgttcgtcatgtttctgagcctgattggtctgctggcaatcgccggcattagactgcaccgcgccgctatctatacagcagagattcataagtccctgtctaccaacctggacgtgacaaattccatcgaacatcaggtgaaggatgtcctgactccactgttcaaaatcattggtgacgaggtcggactgcgaactccacagcggttcaccgacctggtgaagtttatcagcgataagatcaagttcctgaacccagaccgggagtacgactttagggatctgacctggtgcatcaacccccctgaacggatcaagctgaactacgaccagtattgtgcagatgtcgcagccgaggaactgatgaacgccctggtgaattccacactgctggagactaggaccacaaaccagttcctggccgtgtctaagggaaattgccctgggccaactaccatccgcggccagttttcaaatatgagtctgtcactgctggatctgtacctgagccgaggatataacgtgagctccattgtcaccatgacatcccaggggatgtacggcggaacctatctggtgggaaagccagacctgaattccaaagggtctgagctgagtcagccttcaatgtaccgagtgttcgaagtgggcgtcatccgaaacccaggactgggagcacccgtgttccacatgaccaattattttgagcagcccattagcaaggatctgtccaactgtatggtggccctgggtgaactgaaactggctgcactgtgccataggggcgacagtatcacaattccctgtagagggtccggcaagggcgtgtcttttcagctggtgaagctgggcgtctggaaaagccctactgatatgcacagttgggtcccactgtcaaccgacgatcccgtgatcgatagactgtacctgtctagtcatcgcggagtcattaccgacaaccaggctaattgggcagtgcctacaactagaaccgacgataagctgcagaaagagacatgcttccagcaggcctgtaaggggaaaatccaggctctgtgcgagaatctggaatgggctcctctgaaggattctcgcattccaagttatggagtgctgtcagtcaacctgagcctggccgctgaacccaagatcaaaattgcctctggatttgggcctctgatcactcacggtagcggcatggacctgtacaagtccaaccataacaacgtgtactggctgactattccacccatgaaaaacctggctctgggcgtgatcaataccctgaagtggattcctcgcctgaaggtcagcccatacctgtttacagtgcccatcgaggaagccgacgaggattgtcgggctccaacatacctgcccgcagaagtgactggcgacgtgaaactgtcaagcaacctggtcatcctgccaggacaggacctgcagtacgtgctggctacttatgatacctctggagtcgaacatgcagtggtctactacgtgtacagccctggcgggtctttcagttacgtgtatccttttcggctgccaatcaagggtacaccaattgagctgcaggtggaatgcttcacttgggcccagaggctgtggtgcagacacttttgcgtgctggctgattcagagagcgggggtcacctgacccattctggaatggtggggatggaagtcagttgtacagtgaacagggaggacgaagccaatatcgagggaagggcggcccagccggcccaggtgcagctgcagcagagcggcgccgagctggccaggcccggcgccagcgtgaagatgagctgcaaggccagcggctacaccttcaccaggtacaccatgcactgggtgaagcagaggcccggccagggcctggagtggatcggctacatcaaccccagcaggggctacaccaactacaaccagaagttcaaggacaaggccaccctgaccaccgacaagagcagcagcaccgcctacatgcagctgagcagcctgaccagcgaggacagcgccgtgtactactgcgccaggtactacgacgaccactactgcctggactactggggccagggcaccaccctgaccgtgagcagcgccggcggcggcggcagcggcggcggcggcagcggcggcggcggcagccagatcgtgctgacccagagccccgccatcatgagcgccagccccggcgagaaggtgaccatgacctgcagcgccagcagcagcgtgagctacatgaactggtaccagcagaagagcggcaccagccccaagaggtggatctacgacaccagcaagctggccagcggcgtgcccgcccacttcaggggcagcggcagcggcaccagctacagcctgaccatcagcggcatggaggccgaggacgccgccacctactactgccagcagtggagcagcaaccccttcaccttcggcagcggcaccaagctggagatcaacagggccgacaccgcccccaccgcggccgcaagaggatcgcatcaccatcaccatcactgatagtttgtactagtgtgaaatagacatcagaattaagaaaaacgtagggtccaagtggttccccgttatggactcgctatctgtcaaccagatcttataccctgaagttcacctagatagcccgatagttaccaataagatagtagccatcctggagtatgctcgactgtttaaacctgcaggcatgcaagctgatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgaggagctttttgcaaaagcctaggcctccaaaaaagcctcttcactacttctggaatagctcagaggccgcggcctcggcctctgcataaataaaaaaaattagtcagccatggggcggagaatgggcggaactgggcggagttaggggcgggatgggcggagttaggggcgggactatggttgctgactaattgctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccattcgacgctctcccttatgcgactcctgcattaggaagcagcccagtagtaggttgaggccgttgagcaccgccgccgcaaggaatggtctggcttatcgaaattaatacgactcactatagggagaccg